SEQUENCE LISTING (4 sequences)

SEQ ID NO: 1 - protein, 10 aa, Artificial Sequence
  Peptide
NVLHFFNAPL

SEQ ID NO: 2 - protein, 9 aa, Artificial Sequence
  Peptide
ASLDSDPWV

SEQ ID NO: 3 - protein, 10 aa, Artificial Sequence
  Peptide
KLKHYGPGWV

SEQ ID NO: 4 - protein, 9 aa, Artificial Sequence
  Peptide
LLQAEAPRL

(19) United States
(12) Patent Application Publication
(10) Pub. No.: US 2023/0321241 A1
(43) Pub. Date: Oct. 12, 2023

(54) CANCER PEPTIDE VACCINE AND METHOD OF PREPARING THE SAME

(71) Applicant: BrightPath Biotherapeutics Co., Ltd., Kanagawa (JP)

(72) Inventor: Kazuhiko ODAKA, Kurume-shi, Fukuoka (JP)

(73) Assignee: BrightPath Biotherapeutics Co., Ltd., Kanagawa (JP)

(21) Appl. No.: 18/018,380

(22) PCT Filed: Jul. 28, 2021
(86) PCT No.: PCT/JP2021/027832

§ 371 Date: Jan. 27, 2023

(30) Foreign Application Priority Data
Jul. 29, 2020 (JP) 2020-128412

(51) Int. Cl. A61K 39/00; A61K 39/395; A61P 37/04; A61K 9/19

(52) U.S. Cl. CPC A61K 39/4634; A61K 39/3955; A61P 37/04; A61K 9/19; A61K 2039/505

(57) ABSTRACT

The present disclosure comprises a cancer peptide vaccine comprising a peptide of Asn-Val-Leu-His-Phe-Phe-Asn-Ala-Pro-Leu (SEQ ID NO: 1), a peptide of Ala-Ser-Leu-Asp-Ser-Asp-Pro-Trp-Val (SEQ ID NO: 2), a peptide of Lys-Leu-Lys-His-Tyr-Gly-Pro-Gly-Trp-Val (SEQ ID NO: 3), and a peptide of Leu-Leu-Gln-Ala-Glu-Ala-Pro-Arg-Leu (SEQ ID NO: 4), and a method of preparing the same.

### TECHNICAL FIELD

This application claims priority with respect to Japanese Patent Application No. 2020-128412, which is incorporated herein by reference in its entirety.

The present disclosure relates to a cancer peptide vaccine and a method of preparing the same.

### BACKGROUND

About 18 million subjects newly develop cancers every year worldwide, of which about 9.6 million die. In Japan, the predicted number of cancer cases is about 1 million and that of deaths is about 380000 in 2019 (https://ganjoho.jp/reg_stat/statistics/stat/short_pred.html). Main therapeutic methods developed for such cancers include surgery, radiotherapy, chemotherapy, molecular-targeted drugs, antibody drugs, immunotherapy, and cell therapy.

Particularly in recent years, immune checkpoint inhibitors targeting immune checkpoint molecules such as CTLA-4, PD-1, and PD-L1 have been developed (Patent Document 1 or 2). Immunotherapies using these inhibitors have been shown to provide excellent clinical effects that could not be achieved by conventional therapeutic methods, and various immune checkpoint inhibitors and therapeutic methods using these inhibitors are being actively developed. However, immune checkpoint inhibitors do not necessarily provide effective clinical effects in all patients. About 20 to 50% of patients does not respond to immune checkpoint inhibitors and clinical effects are not obtained in some cancers. Therefore, in order to increase the response rate of immune checkpoint inhibitors, combination therapies of an immune checkpoint inhibitor with a drug such as a chemotherapeutic agent or a molecular targeted drug are being actively studied.

One of the drug candidates to be used in combination with an immune checkpoint inhibitor is a cancer vaccine using a tumor antigen peptide, which is also used for immunotherapy. Cell-mediated immunity, especially cytotoxic T cells (called CTLs), play an important role in the elimination of tumor cells by the living body. CTLs are produced as a result of differentiation and proliferation of precursor T cells that have recognized a complex of an antigen peptide on tumor cells, that is, a tumor antigen peptide, and a human leukocyte antigen (HLA) class I antigen, and they attack cancer cells. Therefore, by using a tumor antigen peptide as a drug to induce the CTL activity on cancer cells so that the CTLs attack cancer cells, clinical effects such as tumor shrinkage effect can be obtained.

As such tumor antigen peptides, peptides derived from MAGE-A3 antigen, which was discovered at the Ludwig Institute, or tumor-specific antigens or tumor-related antigens such as EGFRvIII and gp100 have been studied, for example (Patent Documents 3 to 7 and Non-Patent Documents 1 to 26). However, cancer vaccines comprising these peptides alone failed to show sufficient clinical effects and there has still been no approved cancer vaccine. Also, as an example of a combination therapy with an immune checkpoint inhibitor, a combination study of an anti-CTLA-4 antibody and a cancer vaccine comprising a gp100 peptide was carried out, but this study failed to demonstrate effectiveness of the combination therapy (Non-Patent Document 27).

### CITATION LIST

### Patent Documents

Patent Document 1: WO2011/011027
Patent Document 2: WO2004/004771
Patent Document 3: WO99/67288
Patent Document 4: WO00/12701
Patent Document 5: WO02/010369
Patent Document 6: WO2009/038026
Patent Document 7: WO2014/181805

### Non Patent Documents

Non Patent Document 1: Ito, M., et al., Molecular basis of T cell-mediated recognition of pancreatic cancer cells. Cancer Res, 2001. 61(5): p. 2038-46.
Non Patent Document 2: Tamura, M., et al., Identification of cyclophilin B-derived peptides capable of inducing histocompatibility leukocyte antigen-A2-restricted and tumor-specific cytotoxic T lymphocytes. Jpn J Cancer Res, 2001. 92(7): p. 762-7.
Non Patent Document 3: Ito, M., et al., Identification of SART3-derived peptides capable of inducing HLA-A2-restricted and tumor-specific CTLs in cancer patients with different HLA-A2 subtypes. Int J Cancer, 2000. 88(4): p. 633-9.
Non Patent Document 4: Tanaka, S., et al., Peptide vaccination for patients with melanoma and other types of cancer based on pre-existing peptide-specific cytotoxic T-lymphocyte precursors in the periphery. J Immunother, 2003. 26(4): p. 357-66.
Non Patent Document 5: Yoshiyama, K., et al., Personalized peptide vaccination in patients with refractory non-small cell lung cancer. Int J Oncol, 2012. 40(5): p. 1492-500.
Non Patent Document 6: Terazaki, Y., et al., Immunological evaluation of personalized peptide vaccination in refractory small cell lung cancer. Cancer Sci, 2012. 103(4): p. 638-44.
Non Patent Document 7: Noguchi, M., et al., Phase I trial of patient-oriented vaccination in HLA-A2-positive patients with metastatic hormone-refractory prostate cancer. Cancer Sci, 2004. 95(1): p. 77-84.
Non Patent Document 8: Noguchi, M., et al., Immunological monitoring during combination of patient-oriented peptide vaccination and estramustine phosphate in patients with metastatic hormone refractory prostate cancer. Prostate, 2004. 60(1): p. 32-45.
Non Patent Document 9: Noguchi, M., et al., Combination therapy of personalized peptide vaccination and low-dose estramustine phosphate for metastatic hormone refractory prostate cancer patients: an analysis of prognostic factors in the treatment. Oncol Res, 2007. 16(7): p. 341-9.
Non Patent Document 10: Naito, M., et al., Dexamethasone did not suppress immune boosting by personalized peptide vaccination for advanced prostate cancer patients. Prostate, 2008. 68(16): p. 1753-62.
Non Patent Document 11: Uemura, H., et al., Immunological evaluation of personalized peptide vaccination monotherapy in patients with castration-resistant prostate cancer. Cancer Sci, 2010. 101(3): p. 601-8.
Non Patent Document 12: Noguchi, M., et al., Phase II study of personalized peptide vaccination for castration-resistant prostate cancer patients who failed in docetaxel-based chemotherapy. Prostate, 2012. 72(8): p. 834-45.
Non Patent Document 13: Yamada, A., et al., Phase I clinical study of a personalized peptide vaccination available for six different human leukocyte antigen (HLA-A2, -A3, -A11, -A24, -A31 and -A33)-positive patients with advanced cancer. Exp Ther Med, 2011. 2(1): p. 109-117.
Non Patent Document 14: Yamamoto, K., et al., Immunological evaluation of personalized peptide vaccination for patients with pancreatic cancer. Oncol Rep, 2005. 13(5): p. 875-83.
Non Patent Document 15: Yanagimoto, H., et al., Immunological evaluation of personalized peptide vaccination with gemcitabine for pancreatic cancer. Cancer Sci, 2007. 98(4): p. 605-11.
Non Patent Document 16: Yanagimoto, H., et al., A phase II study of personalized peptide vaccination combined with gemcitabine for non-resectable pancreatic cancer patients. Oncol Rep, 2010. 24(3): p. 795-801.
Non Patent Document 17: Sato, Y., et al., Immunological evaluation of peptide vaccination for patients with gastric cancer based on pre-existing cellular response to peptide. Cancer Sci, 2003. 94(9): p. 802-8.
Non Patent Document 18: Mochizuki, K., et al., Immunological evaluation of vaccination with pre-designated peptides frequently selected as vaccine candidates in an individualized peptide vaccination regimen. Int J Oncol, 2004. 25(1): p. 121-31.
Non Patent Document 19: Sato, Y., et al., Immunological evaluation of personalized peptide vaccination in combination with a 5-fluorouracil derivative (TS-1) for advanced gastric or colorectal carcinoma patients. Cancer Sci, 2007. 98(7): p. 1113-9.
Non Patent Document 20: Hattori, T., et al., Immunological evaluation of personalized peptide vaccination in combination with UFT and UZEL for metastatic colorectal carcinoma patients. Cancer Immunol Immunother, 2009. 58(11): p. 1843-52.
Non Patent Document 21: Suekane, S., et al., Phase I trial of personalized peptide vaccination for cytokine-refractory metastatic renal cell carcinoma patients. Cancer Sci, 2007. 98(12): p. 1965-8.
Non Patent Document 22: Matsumoto, K., et al., A phase I study of personalized peptide vaccination for advanced urothelial carcinoma patients who failed treatment with methotrexate, vinblastine, adriamycin and cisplatin. BJU Int, 2010. 108(6): p. 831-8.
Non Patent Document 23: Tsuda N., et al., Vaccination with predesignated or evidence-based peptides for patients with recurrent gynecologic cancers. J Immunother, 2004. 27: p. 60-72
Non Patent Document 24: Yajima, N., et al., Immunologic evaluation of personalized peptide vaccination for patients with advanced malignant glioma. Clin Cancer Res, 2005. 11(16): p. 5900-11.
Non Patent Document 25: Takahashi, R., et al., Phase II study of personalized peptide vaccination for refractory bone and soft tissue sarcoma patients. Cancer Sci, 2013. 104(10): p. 1285-94.
Non Patent Document 26: Yoshida, K., et al., Characteristics of severe adverse events after peptide vaccination for advanced cancer patients: Analysis of 500 cases. Oncol Rep, 2011. 25(1): p. 57-62.
Non Patent Document 27: F. Stephen Hodi, M. D., et al., Improved Survival with Ipilimumab in Patients with Metastatic Melanoma, N Engl J Med. 2010 Aug. 19; 363(8): 711-723

### SUMMARY

### Problem to be Solved

An object of the present invention is to provide a cancer peptide vaccine and a method of preparing the same.

### Means to Solve Problem

The inventors have diligently investigated a cancer peptide vaccine, particularly a cancer peptide vaccine suitable for combination use with an immune checkpoint inhibitor. Then, the inventors have found that a cancer peptide vaccine comprising the four peptides of SEQ ID NOs: 1 to 4 induces CTLs in human subjects, and exerts an excellent clinical effect particularly in combination with an immune checkpoint inhibitor. Also, the inventors have found a formulation of the cancer peptide vaccine comprising the four peptides and a method of preparing the cancer peptide vaccine that enable stable production and supply of the cancer peptide vaccine.

In an aspect, the present disclosure provides a cancer peptide vaccine comprising a peptide of Asn-Val-Leu-His-Phe-Phe-Asn-Ala-Pro-Leu (SEQ ID NO: 1), a peptide of Ala-Ser-Leu-Asp-Ser-Asp-Pro-Trp-Val (SEQ ID NO: 2), a peptide of Lys-Leu-Lys-His-Tyr-Gly-Pro-Gly-Trp-Val (SEQ ID NO: 3), and a peptide of Leu-Leu-Gln-Ala-Glu-Ala-Pro-Arg-Leu (SEQ ID NO: 4).

In a further aspect, the present disclosure provides a method of preparing a cancer peptide vaccine, comprising adding a peptide of Asn-Val-Leu-His-Phe-Phe-Asn-Ala-Pro-Leu (SEQ ID NO: 1) to an aqueous solution comprising glycine, wherein the cancer peptide vaccine comprises the peptide of Asn-Val-Leu-His-Phe-Phe-Asn-Ala-Pro-Leu (SEQ ID NO: 1), a peptide of Ala-Ser-Leu-Asp-Ser-Asp-Pro-Trp-Val (SEQ ID NO: 2), a peptide of Lys-Leu-Lys-His-Tyr-Gly-Pro-Gly-Trp-Val (SEQ ID NO: 3), and a peptide of Leu-Leu-Gln-Ala-Glu-Ala-Pro-Arg-Leu (SEQ ID NO: 4).

### Effect of Invention

The cancer peptide vaccine of the present disclosure induces CTLs in human subjects and exerts an excellent clinical effect in combination with an immune checkpoint inhibitor. Also, the preparation method of the present disclosure provides a cancer peptide vaccine in which the solubility and stability of its active ingredient, i.e., a cancer antigen peptide, are guaranteed, and thus enables stable production and supply of pharmaceutical products.

### BRIEF DESCRIPTION OF DRAWINGS

FIG. 1 shows the results of analyzing the tumor shrinkage effects in patients who received a peptide cocktail preparation comprising the peptides of SEQ ID NOs: 1 to 4.

FIG. 2 shows the results of analyzing the number of IFN-γ-producing cells that responded to each of the peptides of SEQ ID NOs: 1 to 4 in peripheral blood mononuclear cells of patients who received the peptide cocktail preparation comprising the peptides.

### DETAILED DESCRIPTION OF EMBODIMENTS

The cancer peptide vaccine of the present disclosure comprises the four peptides of SEQ ID NOs: 1 to 4 as shown below.

(SEQ ID NO: 1)
Asn-Val-Leu-His-Phe-Phe-Asn-Ala-Pro-Leu
(SEQ ID NO: 2)
Ala-Ser-Leu-Asp-Ser-Asp-Pro-Trp-Val
(SEQ ID NO: 3)
Lys-Leu-Lys-His-Tyr-Gly-Pro-Gly-Trp-Val
(SEQ ID NO: 4)
Leu-Leu-Gln-Ala-Glu-Ala-Pro-Arg-Leu

These peptides are peptides derived from tumor antigen proteins expressed by cancer cells (herein also referred to as tumor antigen peptides), and they are recognized by HLA class I-restricted CTLs and induce cytotoxic activity to cancer cells. In the present disclosure, a cancer peptide vaccine is synonymous with a peptide vaccine for treating cancer and it is a medicament to treat cancer by inducing an immune response against cancer cells using a tumor antigen peptide as its active ingredient. In the present disclosure, the term “peptide” is used in the sense that it encompasses a pharmaceutically acceptable salt thereof, unless it is inappropriate in context.

The “pharmaceutically acceptable salt” in the present disclosure includes acetate, hydrochloride, hydrobromide, sulfate, hydroiodide, nitrate, phosphate, citrate, oxalate, formate, propionate, benzoate, trifluoroacetate, maleate, tartrate, methanesulfonate, benzenesulfonate, paratoluenesulfonate, sodium salt, potassium salt, calcium salt, magnesium salt, ammonium salt, triethylammonium salt, triethanolammonium salt, pyridinium salt, and diisopropylammonium salt.

A cancer peptide vaccine may be a single composition comprising the four peptides of SEQ ID NOs: 1 to 4, or a kit comprising two or more compositions each comprising one of the peptides of SEQ ID NOs: 1 to 4, or a combination of two or three of the peptides of SEQ ID NOs: 1 to 4. In an embodiment, the cancer peptide vaccine is a composition comprising the peptides of SEQ ID NOs: 1 to 4. In another embodiment, the cancer peptide vaccine is a kit comprising four compositions each comprising one of the peptides of SEQ ID NOs: 1 to 4.

Peptides can be produced by conventional methods, for example those described in references such as PeptideSynthesis, Interscience, NewYork, 1966; The Proteins, Vol. 2, Academic Press Inc, NewYork, 1976; Basics and Experiments of Peptide Synthesis, Maruzen Co., Ltd. 1985; Development of Pharmaceuticals Vol. 14, Peptide Synthesis, Hirokawa Shoten, 1991, but methods for producing peptides are not limited to these methods and different known methods are widely available. The purification and recovery of the peptides can be carried out by a known method such as chromatography including gel chromatography, ion column chromatography, or affinity chromatography, or fractionating process based on the solubility difference using an agent such as ammonium sulfate or alcohol. It is also possible to prepare a polyclonal or monoclonal antibody specific to the peptide based on the amino acid sequence of the peptide and use the antibody thus prepared for specific adsorption and recovery of the peptide. The peptide thus prepared is usually recovered as a salt of the peptide with a counter ion such as acetic acid, hydrochloric acid, sodium or trifluoroacetic acid. Such a salt can be used as a drug substance to prepare a cancer peptide vaccine. When the mass of a peptide is referred in the present disclosure, it means the mass of the peptide in free form, not salt form.

The cancer peptide vaccine preferably comprises one or more pharmaceutically acceptable additives in addition to the peptide. Pharmaceutically acceptable additives include buffering agents, antioxidants, preservatives, excipients, suspending agents, tonicity agents, chelating agents, and surfactants, and specific examples thereof include buffering agents such as phosphoric acid, citric acid, and other organic acids; antioxidants such as ascorbic acid and methionine; preservatives such as octadecyldimethylbenzyl ammonium chloride, hexamethonium chloride, benzalkonium chloride, and benzethonium chloride; polypeptides; proteins such as serum albumin, gelatin, and immunoglobulins; hydrophilic polymers such as polyvinylpyrrolidone; amino acids such as glycine, glutamine, asparagine, histidine, arginine, and lysine; monosaccharides, disaccharides, and other carbohydrates including glucose, mannose, and dextrins; chelating agents such as EDTA; sugars such as sucrose, mannitol, trehalose, and sorbitol; salt-forming counterions such as sodium; metal complexes (eg, Zn-protein complexes); and nonionic surfactants such as TWEEN™, PLURONICS™, and polyethylene glycol (PEG).

In an embodiment, the cancer peptide vaccine of the present disclosure comprises glycine. Since the solubility of the peptide of SEQ ID NO: 1 in aqueous solutions is low, it is preferred to improve the solubility by formulation design. For improving peptide solubility in aqueous solutions, sodium hydrogencarbonate has been used. However, there are some difficulties with sodium hydrogencarbonate, especially when the peptide preparation is a lyophilized preparation, since sodium hydrogencarbonate produces water and prevents accurate determination of the water content in the lyophilized preparation, and also the pH of an aqueous solution prepared from the preparation becomes basic and the peptide is readily oxidized. As shown in Examples, by using glycine, the peptide of SEQ ID NO: 1 can be dissolved in an aqueous solution and it does not precipitate after dissolution, that is, the stability after dissolution is improved. The amount of glycine comprised in the cancer peptide vaccine is not particularly limited to, but preferably, an amount such that the mass ratio of the peptide of SEQ ID NO: 1 to glycine is 0.3 to 30, 0.4 to 28, 0.6 to 3.0, 0.7 to 2.9, or 0.8 to 2.8. In the present disclosure, the term “glycine” is used in the sense that it encompasses a pharmaceutically acceptable salt thereof, unless it is inappropriate in context. In an embodiment, glycine is glycine hydrochloride. When the mass of glycine is referred in the present disclosure, it means the mass of glycine in free form.

In an embodiment, the cancer peptide vaccine of the present disclosure comprises citric acid. The amount of citric acid comprised in the peptide vaccine is not particularly limited to, but preferably, an amount such that the mass ratio of each peptide of SEQ ID NOs: 2 to 4 to citric acid is 0.1 to 20 or 0.3 to 10. In the present disclosure, the term “citric acid” is used in the sense that it encompasses a pharmaceutically acceptable salt, unless it is inappropriate in context. When the mass of citric acid is referred in the present disclosure, it means the mass of citric acid in free form.

In an embodiment, the cancer peptide vaccine of the present disclosure comprises trehalose. The amount of trehalose comprised in the cancer peptide vaccine is not particularly limited to, but preferably, an amount such that the mass ratio of each peptide to trehalose is 0.01 to 1 or 0.02 to 0.3. When the mass of trehalose is referred in the present disclosure, it means the mass of trehalose anhydride.

In a further embodiment, the cancer peptide vaccine of the present disclosure comprises glycine and trehalose, or glycine, citric acid, and trehalose.

In a further embodiment, the cancer peptide vaccine of the present disclosure comprises the following compositions:
a composition comprising the peptide of SEQ ID NO: 1, glycine and trehalose, wherein the mass ratio of the peptide of SEQ ID NO: 1 to glycine is 2.67 and the mass ratio of the peptide of SEQ ID NO: 1 to trehalose is 0.15;
a composition comprising the peptide of SEQ ID NO: 2, citric acid and trehalose, wherein the mass ratio of the peptide of SEQ ID NO: 2 to citric acid is 1.04 and the mass ratio of the peptide of SEQ ID NO: 2 to trehalose is 0.11;
a composition comprising the peptide of SEQ ID NO: 3, citric acid and trehalose, wherein the mass ratio of the peptide of SEQ ID NO: 3 to citric acid is 5.21 and the mass ratio of the peptide of SEQ ID NO: 3 to trehalose is 0.11;
a composition comprising the peptide of SEQ ID NO: 4, citric acid and trehalose, wherein the mass ratio of the peptide of SEQ ID NO: 4 to citric acid is 5.21 and the mass ratio of the peptide of SEQ ID NO: 4 to trehalose is 0.11.

In a further embodiment, the cancer peptide vaccine of the present disclosure comprises a composition comprising the peptides of SEQ ID NOs: 1 to 4, glycine and trehalose, wherein the mass ratio of the peptide of SEQ ID NO: 1 to glycine is 0.8 and the mass ratio of each peptide to trehalose is 0.03.

In a further embodiment, the cancer peptide vaccine of the present disclosure comprises a composition comprising the peptides of SEQ ID NOs: 1 to 4 and glycine, wherein the mass ratio of each peptide to glycine is 0.8.

The dosage form of the cancer peptide vaccine is not particularly limited to, but preferably, an aqueous solution preparation or a lyophilized preparation, and more preferably a lyophilized preparation in view of peptide stability. The cancer peptide vaccine may be a preparation (eg, an aqueous solution preparation or a lyophilized preparation) comprising the four peptides of SEQ ID NOs: 1 to 4, or may be a kit comprising two or more preparations (eg, aqueous solution preparations or lyophilized preparations) each comprising one of the peptides of SEQ ID NOs: 1 to 4, or a combination of two or three of the peptides of SEQ ID NOs: 1 to 4. Preferably, the cancer peptide vaccine is a lyophilized preparation comprising the four peptides of SEQ ID NOs: 1 to 4, and more preferably, a lyophilized preparation comprising the four peptides of SEQ ID NOs: 1 to 4 and glycine.

The cancer peptide vaccine of the present disclosure is administered in the form of an aqueous solution. When the cancer peptide vaccine is an aqueous solution preparation, it can be administered to a patient as it is, and when it is a lyophilized preparation, it is administered after being dissolved in a pharmaceutically acceptable solvent (eg, purified water or water for injection). When the cancer peptide vaccine is a kit comprising two or more preparations, aqueous solutions each comprising a peptide may be administered separately, or may be mixed and then administered. In an embodiment, among the aqueous solutions at the time of administration of the cancer peptide vaccine, the aqueous solution comprising the peptide of SEQ ID NO: 1 has a pH of 3.5 to 5.0, and the aqueous solution comprising each peptide of SEQ ID NOs: 2 to 4 has a pH of 3.0 to 8.0 or a pH of 4.0 to 7.0. In another embodiment, the aqueous solution at the time of administration of the cancer peptide vaccine comprises the four peptides of SEQ ID NOs: 1 to 4 and has a pH of 3.5 to 8.2. In a further embodiment, the aqueous solution at the time of administration of the cancer peptide vaccine comprises the four peptides of SEQ ID NOs: 1 to 4 and has a pH of 3.5 to 5.0. In a further embodiment, the aqueous solution at the time of administration of the cancer peptide vaccine comprises the four peptides of SEQ ID NOs: 1 to 4 and has a pH of 6.0 to 8.0. In an example, the cancer peptide vaccine is a lyophilized preparation, and an aqueous solution after dissolution in a pharmaceutically acceptable solvent has the above pH.

The cancer peptide vaccine of the present disclosure can be prepared by adding a peptide to an aqueous solution comprising a pharmaceutically acceptable additive, and optionally adjusting pH of the resulting solution, to provide an aqueous solution comprising the peptide, and optionally freeze-drying the aqueous solution thus obtained. The pH of the solution can be adjusted with, for example, hydrochloric acid (HCl), sodium hydroxide (NaOH), or a buffer such as a phosphate buffer or a histidine buffer. If necessary, the aqueous solution comprising the peptide may be sterilized by filtration, filled in a container such as a glass vial, and provided as an aqueous solution, or may be subjected to lyophilization after filled in a container and provided as a lyophilized preparation.

When the peptide is added to an aqueous solution comprising a pharmaceutically acceptable additive, the peptides of SEQ ID NOs: 1 to 4 may be added to separate aqueous solutions, or some of the peptides of SEQ ID NOs: 1 to 4 may be added to one aqueous solution in combination, or all the peptides of SEQ ID NOs: 1 to 4 may be added to a single aqueous solution. When two or more aqueous solutions comprising the peptides are obtained, these aqueous solutions may be separately lyophilized to form a kit comprising two or more lyophilized preparations, or the components after lyophilization may be mixed to form a single lyophilized preparation.

When the peptide of SEQ ID NO: 1 is added to an aqueous solution comprising a pharmaceutically acceptable additive, it is preferable that the peptide of SEQ ID NO: 1 is added so that the concentration of the peptide of SEQ ID NO: 1 in the aqueous solution after the addition is 20 mg/mL or less, preferably 3 to 20 mg/mL, more preferably 3 mg to 10 mg/mL. In an embodiment, the peptide of SEQ ID NO: 1 is added to an aqueous solution comprising glycine. The aqueous solution comprising glycine preferably has a pH of 3.5 to 5.0. The mass ratio of the peptide of SEQ ID NO: 1 to glycine in the aqueous solution after the addition is preferably 0.3 to 30, 0.4 to 28, 0.6 to 3.0, 0.7 to 2.9, or 0.8 to 2.8. In a further embodiment, the peptide of SEQ ID NO: 1 is added to an aqueous solution comprising trehalose in addition to glycine. The mass ratio of the peptide of SEQ ID NO: 1 to trehalose in the aqueous solution after the addition is, for example, 0.01 to 1 or 0.02 to 0.3. The pH of the aqueous solution after the addition of the peptide of SEQ ID NO: 1 is preferably adjusted to pH 3.5 to 5.0, if necessary.

When any of the peptides of SEQ ID NOs: 2 to 4 is added to an aqueous solution comprising a pharmaceutically acceptable additive, the concentration of the peptide in the aqueous solution after the addition is not particularly limited to, and can be, 20 to 200 mg/mL for example. It is preferable that the peptide is added so that the concentration of the peptide in the aqueous solution after the addition is 20 mg/mL or less, preferably 3 to 20 mg/mL, more preferably 3 mg to 10 mg/mL. In an embodiment, each of the peptides of SEQ ID NOs: 2 to 4 is added to an aqueous solution comprising citric acid. The aqueous solution comprising citric acid has a pH, for example, of 3.0 to 8.0 or 4.0 to 7.0. The mass ratio of each peptide to citric acid in the aqueous solution after the addition is, for example, 0.1 to 20 or 0.3 to 10. In a further embodiment, each of the peptides of SEQ ID NOs: 2 to 4 is added to an aqueous solution comprising trehalose in addition to citric acid. The mass ratio of the peptide to trehalose in the aqueous solution after the addition is, for example, 0.01 to 1 or 0.02 to 0.3. The pH of the aqueous solution after the addition of any of the peptides of SEQ ID NOs: 2 to 4 is preferably adjusted to pH 3.0 to 8.0 or pH 4.0 to 7.0, if necessary.

When the four peptides of SEQ ID NOs: 1 to 4 are added to a single aqueous solution comprising a pharmaceutically acceptable additive, it is preferable that the peptides are added so that the concentration of each peptide is 20 mg/mL or less, preferably 3 to 20 mg/mL, more preferably 3 mg to 10 mg/mL. The aqueous solution comprising a pharmaceutically acceptable additive preferably has a pH of 3.5 to 5.0. In an embodiment, these four peptides are added to an aqueous solution comprising glycine. The amount of glycine in relation to the peptide of SEQ ID NO: 1 in the aqueous solution after the addition is preferably an amount such that the mass ratio of the peptide of SEQ ID NO: 1 to glycine is 0.3 to 30, 0.4 to 28, 0.6 to 3.0, 0.7 to 2.9, or 0.8 to 2.8. In a further embodiment, these four peptides are added to an aqueous solution comprising citric acid in addition to glycine. The mass ratio of each peptide to citric acid in the aqueous solution after the addition is, for example, 0.1 to 20 or 0.3 to 10. In a further embodiment, these four peptides are added to an aqueous solution comprising trehalose in addition to glycine or in addition to glycine and citric acid. The mass ratio of each peptide to trehalose in the aqueous solution after the addition is, for example, 0.01 to 1 or 0.02 to 0.3. The pH of the aqueous solution after the addition of the peptides of SEQ ID NOs: 1 to 4 is preferably adjusted to a pH of 3.5 to 8.2, if necessary. The aqueous solution after the addition of the peptides of SEQ ID NOs: 1 to 4 may have a pH of 3.5 to 5.0 or 6.0 to 8.0.

In a further embodiment,
the peptide of SEQ ID NO: 1 is added to an aqueous solution comprising glycine and trehalose, wherein the aqueous solution comprising glycine and trehalose has a pH of 3.5, and wherein the mass ratio of the peptide of SEQ ID NO: 1 to glycine is 2.67 and the mass ratio of the peptide of SEQ ID NO: 1 to trehalose is 0.15 in the aqueous solution after the addition, and the pH of the aqueous solution after the addition is adjusted to 3.5 if necessary;
the peptide of SEQ ID NO: 2 is added to an aqueous solution comprising citric acid and trehalose, wherein the aqueous solution comprising citric acid and trehalose has a pH of 4, and wherein the mass ratio of the peptide of SEQ ID NO: 2 to citric acid is 1.04 and the mass ratio of the peptide of SEQ ID NO: 2 to trehalose is 0.11 in the aqueous solution after the addition, and the pH of the aqueous solution after the addition is adjusted to 4 if necessary;
the peptide of SEQ ID NO: 3 is added to an aqueous solution comprising citric acid and trehalose, wherein the aqueous solution comprising citric acid and trehalose has a pH of 7, and wherein the mass ratio of the peptide of SEQ ID NO: 3 to citric acid is 5.21 and the mass ratio of the peptide of SEQ ID NO: 3 to trehalose is 0.11 in the aqueous solution after the addition, and the pH of the aqueous solution after the addition is adjusted to 7 if necessary;
the peptide of SEQ ID NO: 4 is added to an aqueous solution comprising citric acid and trehalose, wherein the aqueous solution comprising citric acid and trehalose has a pH of 6, and wherein the mass ratio of the peptide of SEQ ID NO: 4 to citric acid is 5.21 and the mass ratio of the peptide of SEQ ID NO: 4 to trehalose is 0.11 in the aqueous solution after the addition, and the pH of the aqueous solution after the addition is adjusted to 6 if necessary; and
the concentration of each peptide after the addition is 10 mg/mL.

In a further embodiment,
the peptides of SEQ ID NOs: 1 to 4 are added to an aqueous solution comprising glycine and trehalose, wherein the aqueous solution comprising glycine and trehalose has a pH of 3.5, and wherein the mass ratio of the peptide of SEQ ID NO: 1 to glycine is 0.8 and the mass ratio of each peptide to trehalose is 0.03 in the aqueous solution after the addition, the concentration of each peptide after the addition is 3 mg/mL, and the pH of the aqueous solution after the addition is adjusted to 3.5 if necessary.

In a further embodiment,
the peptides of SEQ ID NOs: 1 to 4 are added to an aqueous solution comprising glycine, wherein the aqueous solution comprising glycine has a pH of 3.5, and wherein the mass ratio of the peptide of SEQ ID NO: 1 to glycine is 0.8 in the aqueous solution after the addition, the concentration of each peptide after the addition is 3 mg/mL, and the pH of the aqueous solution after the addition is adjusted to 6.0 to 8.0 if necessary.

Cancers to be treated by the cancer peptide vaccine of the present disclosure are not particularly limited to any specific cancers. Examples of cancers include prostate cancer, pancreatic cancer, colon cancer, lung cancer (including non-small cell lung cancer), hematopoietic tumor, brain tumor, uterine cancer, cervical cancer, gastric cancer, and melanoma (including malignant melanoma), thyroid cancer, liver cancer, and esophageal cancer. Preferably, the cancer peptide vaccine of the present disclosure is used for prostate cancer, brain tumor, melanoma (including malignant melanoma), or lung cancer (including non-small cell lung cancer). Particularly preferably, the cancer peptide vaccine of the present disclosure is used for melanoma (including malignant melanoma) or lung cancer (including non-small cell lung cancer).

The cancer peptide vaccine may further comprise an adjuvant or may be administered with an adjuvant. As an adjuvant, incomplete Freund's adjuvant (eg, ISA-51, SEPPIC) and polysaccharides such as pullulan, which emulsify the peptide aqueous solution and enhance the retention of the peptide at the administration site, and other substances having an immunopotentiating effect such as complete Freund's adjuvant, BCG, alum, GM-CSF, IL-2, and CpG can be used. Preferably, the adjuvant is GM-CSF.

The cancer peptide vaccine is usually administered intradermally or subcutaneously in patients. This is because peptides are rapidly decomposed when administered by intravenous injection, for example, and cannot sufficiently induce immune responses. This is also because CTLs that show cytotoxic activity can be efficiently activated when peptides are administered intradermally or subcutaneously, since antigen-presenting cells that capture antigens and present them on the cell surface via HLA molecules to activate T cells such as CTLs are present in the intradermal or subcutaneous region. The administration site is not particularly limited to, but preferably the vicinity of the lymph node as close to the cancer lesion as possible from the beginning of administration, and examples thereof include the upper arm and thigh. Also, when it becomes difficult to administer the vaccine due to inflammation or other side effects at the administration site, the vaccine may be administered to a different site (eg, abdomen).

The cancer peptide vaccine is administered to a patient in an amount capable of exerting a desired effect (herein also referred to as an “effective amount”). The amount to be administered is not particularly limited to any specific amounts as long as it can be administered intradermally or subcutaneously, but preferably, 0.1 mg or more, preferably 0.1 mg to 3 mg, more preferably 1 mg to 3 mg for each peptide as a mass of the dry powder of the peptide.

The frequency of administration of the peptide may be any number as long as an immune response is obtained, and the peptide may be administered once a day or every 2 days, 3 days, 4 days, 5 days, or 6 days, or once a week or every 2 weeks, 3 weeks, 4 weeks, 5 weeks, or 6 weeks. The frequency of administration may be changed during the administration. For example, the peptide may be administered once a week four times and then administered once every 3 to 4 weeks thereafter. The number of administration of the peptide is, for example, at least 8 times, preferably 16 times or more. There is no upper limit to the number of administration as long as the patient can tolerate the administration. The clinical trials described in Examples performed the peptide administration up to 84 times, and thus the peptide can be administered at least up to that number.

Once a cancer peptide vaccine is administered to a patient, CTLs against the administered peptides are activated in the patient's body, and thereby cancer cells are eliminated and clinical effects are obtained.

The cancer peptide vaccine may be used in combination with one or more other antitumor agents or therapies. Cancer cells of a patient form a heterogeneous population and comprise cells that cannot be eliminated by immune responses and those that are resistant to antitumor agents or hormone therapy or other therapies. The combination use of the cancer peptide vaccine of the present disclosure and a different antitumor agent or therapy improves a clinical effect such as reduction of cancer lesions or extension of survival time.

In an embodiment, the antitumor agent is an immune checkpoint inhibitor. Examples of immune checkpoint inhibitors inhibit the immunosuppressive effects of cancer cells or antigen-presenting cells. Immune checkpoint inhibitors include, for example, agents (eg, antibodies) against the following molecules: CTLA-4 (eg, ipilimumab, tremelimumab), PD-1 (eg, nivolumab, pembrolizumab, AMP-224, AMP-514 (MEDI0680), pidilizumab (CT-011), LAG-3 (eg, IMP-321, BMS-986016), BTLA, KIR (eg, IPH2101), TIM-3, PD-L1 (eg, durvalumab (MEDI4736), MPDL3280A, BMS-936559, Avelumab (MSB0010718C)), PD-L2, B7-H3 (eg, MGA-271), B7-H4, HVEM, GAL9, CD160, VISTA, BTNL2, TIGIT, PVR, BTN1A1, BTN2A2, BTN3A2, and CSF1-R. In a preferred embodiment, the immune checkpoint inhibitor is an agent against PD-1, such as an anti-PD-1 antibody (eg, nivolumab, pembrolizumab, AMP-514 (MEDI0680), pidirizumab (CT-011)).

Examples of antitumor agents also include alkylating agents, antimetabolites, plant alkaloids, topoisomerase inhibitors, microtubule polymerization inhibitors, and molecular targeting agents, and specifically, 5-FU, estramustine, docetaxel, temozolomide, cisplatin, mitoxantrone, gemzar, and rituximab. Examples of therapies include surgery, radiation therapy, hormone therapy (steroids such as dexamethasone, prednisolone, estrogen, and progesterone, and analogs such as leuprorelin).

In the present disclosure, the combination use encompasses simultaneous use and sequential use in any order in the treatment of cancer in one patient. Since the cancer peptide vaccine activates cells of the blood system such as CTLs and thereby exerts its effect, the cancer peptide vaccine is preferably used in combination with other antitumor agents or therapies without affecting the activation of the hematopoietic system and immune responses. For example, the cancer peptide vaccine may be administered after the lymphocyte count has recovered after administration of a different antitumor agent (for example, to 1000 cells/mL or more), or a different antitumor agent or therapy may be used after the administration of the cancer peptide vaccine. Alternatively, a different antitumor agent or therapy may be used during the cancer peptide vaccine is administered as long as the agent or therapy does not decrease the white blood count or lymphocyte count.

In a further aspect, the present disclosure provides a method for treating cancer, comprising administering to a patient a cancer peptide vaccine comprising a peptide of Asn-Val-Leu-His-Phe-Phe-Asn-Ala-Pro-Leu (SEQ ID NO: 1), a peptide of Ala-Ser-Leu-Asp-Ser-Asp-Pro-Trp-Val (SEQ ID NO: 2), a peptide of Lys-Leu-Lys-His-Tyr-Gly-Pro-Gly-Trp-Val (SEQ ID NO: 3), and a peptide of Leu-Leu-Gln-Ala-Glu-Ala-Pro-Arg-Leu (SEQ ID NO: 4).

In a further aspect, the present disclosure provides use of a peptide of Asn-Val-Leu-His-Phe-Phe-Asn-Ala-Pro-Leu (SEQ ID NO: 1), a peptide of Ala-Ser-Leu-Asp-Ser-Asp-Pro-Trp-Val (SEQ ID NO: 2), a peptide of Lys-Leu-Lys-His-Tyr-Gly-Pro-Gly-Trp-Val (SEQ ID NO: 3), and a peptide of Leu-Leu-Gln-Ala-Glu-Ala-Pro-Arg-Leu (SEQ ID NO: 4) for the manufacture of a cancer peptide vaccine.

An exemplary embodiments of the present invention is described below.
[1] A cancer peptide vaccine comprising a peptide of Asn-Val-Leu-His-Phe-Phe-Asn-Ala-Pro-Leu (SEQ ID NO: 1), a peptide of Ala-Ser-Leu-Asp-Ser-Asp-Pro-Trp-Val (SEQ ID NO: 2), a peptide of Lys-Leu-Lys-His-Tyr-Gly-Pro-Gly-Trp-Val (SEQ ID NO: 3), and a peptide of Leu-Leu-Gln-Ala-Glu-Ala-Pro-Arg-Leu (SEQ ID NO: 4).
[2] The cancer peptide vaccine according to item [1], further comprising glycine.
[3] The cancer peptide vaccine according to item [1] or [2], wherein the mass ratio of the peptide of SEQ ID NO: 1 to glycine is 0.3 to 30.
[4] The cancer peptide vaccine according to any one of items [1] to [3], which is a lyophilized preparation.
[5] The cancer peptide vaccine according to any one of items [1] to [4], which is used in combination with an immune checkpoint inhibitor.
[6] The cancer peptide vaccine according to item [5], wherein the immune checkpoint inhibitor is an anti-PD-1 antibody.
[7] The cancer peptide vaccine according to item [6], wherein the anti-PD-1 antibody is pembrolizumab.
[8] A method of preparing a cancer peptide vaccine, comprising adding a peptide of Asn-Val-Leu-His-Phe-Phe-Asn-Ala-Pro-Leu (SEQ ID NO: 1) to an aqueous solution comprising glycine, wherein the cancer peptide vaccine comprises the peptide of Asn-Val-Leu-His-Phe-Phe-Asn-Ala-Pro-Leu (SEQ ID NO: 1), a peptide of Ala-Ser-Leu-Asp-Ser-Asp-Pro-Trp-Val (SEQ ID NO: 2), a peptide of Lys-Leu-Lys-His-Tyr-Gly-Pro-Gly-Trp-Val (SEQ ID NO: 3), and a peptide of Leu-Leu-Gln-Ala-Glu-Ala-Pro-Arg-Leu (SEQ ID NO: 4).
[9] The method according to item [8], wherein the peptide of SEQ ID NO: 1 is added to the aqueous solution comprising glycine so as to provide an aqueous solution having the mass ratio of the peptide of SEQ ID NO: 1 to glycine of 0.3 to 30.
[10] The method according to item [8] or [9], wherein the aqueous solution comprising glycine has a pH of 3.5 to 5.0.
[11] The method according to any one of items [8] to [10], wherein the peptide of SEQ ID NO: 1 is added to the aqueous solution comprising glycine so as to provide an aqueous solution having a peptide concentration of 20 mg/mL or less.
[12] The method according to any one of items [8] to [11], which comprises providing an aqueous solution comprising the peptide of SEQ ID NO: 1, an aqueous solution comprising the peptide of SEQ ID NO: 2, an aqueous solution comprising the peptide of SEQ ID NO: 3, and an aqueous solution comprising the peptide of SEQ ID NO: 4, separately.
[13] The method according to item [12], further comprising lyophilizing each of the aqueous solutions.
[14] The method according to any one of items [8] to [11], which comprises providing an aqueous solution comprising the peptide of SEQ ID NO: 1, the peptide of SEQ ID NO: 2, the peptide of SEQ ID NO: 3, and the peptide of SEQ ID NO: 4.
[15] The method according to item [14], comprising adjusting the pH of the aqueous solution comprising the peptides to a pH of 3.5 to 8.2.
[16] The method according to item [14] or [15], further comprising lyophilizing the aqueous solution.
[17] A method for treating cancer, comprising administering to a patient an effective amount of the cancer peptide vaccine according to any one of items [1] to [7].
[18] The method according to item [17], further comprising administering an immune checkpoint inhibitor.
[19] The method according to item [18], wherein the immune checkpoint inhibitor is an anti-PD-1 antibody.
[20] The method according to item [19], wherein the anti-PD-1 antibody is pembrolizumab.

The above description does not limit the present invention, and a person skilled in the art can make various modifications within the scope of the present disclosure. The present invention will be described in more detail with reference to the following examples, although it is not limited to these examples.

### EXAMPLES

### (Test Example 1) Examination of Peptide Solubility

The solubility of a peptide drug substance (purity 95 w/w % or more, PPL) was examined for the peptides Asn-Val-Leu-His-Phe-Phe-Asn-Ala-Pro-Leu (SEQ ID NO: 1), Ala-Ser-Leu-Asp-Ser-Asp-Pro-Trp-Val (SEQ ID NO: 2), Lys-Leu-Lys-His-Tyr-Gly-Pro-Gly-Trp-Val (SEQ ID NO: 3), and Leu-Leu-Gln-Ala-Glu-Ala-Pro-Arg-Leu (SEQ ID NO: 4), respectively. First, 10 mM citrate buffers (Citrate) or phosphate buffers (Phosphate) containing 270 mM trehalose from pH 3.0 to pH 8.0 were prepared and the peptide drug substance was added to each buffer so as to provide a predetermined peptide concentration. The mixture was stirred and allowed to stand at room temperature for 24 hours to evaluate whether the peptide drug substance was dissolved. Table 1 shows the concentrations of the peptides dissolved and the results of the evaluation.

TABLE 1

| | SEQ ID NO: 1 | | | SEQ ID NO: 2 | | | SEQ ID NO: 3 | | | SEQ ID NO: 4 | | |
|---|---|---|---|---|---|---|---|---|---|---|---|---|
| | buffer | final conc. | | buffer | final conc. | | buffer | final conc. | | buffer | final conc | |
| pH | (mL) | (mg/mL) | result | (mL) | (mg/mL) | result | (mL) | (mg/mL) | result | (mL) | (mg/mL) | result |
| 10 mM Citrate | 15 | 20 | white | 1.5 | 66.67 | dissolved | 0.5 | 200 | dissolved | 5 | 20 | dissolved |
| pH 3.0 | | | turbid | | | | | | | | | |
| 10 mM Citrate | 15 | 20 | white | 1.5 | 66.67 | dissolved | 0.5 | 200 | dissolved | 5 | 20 | dissolved |
| pH 3.5 | | | turbid | | | | | | | | | |
| 10 mM Citrate | 15 | 20 | white | 1 | 100 | dissolved | 0.5 | 200 | dissolved | 3.5 | 28.6 | dissolved |
| pH 4.0 | | | turbid | | | | | | | | | |
| 10 mM Citrate | 15 | 20 | white | 1 | 100 | dissolved | 0.5 | 200 | dissolved | 5 | 20 | dissolved |
| pH 5.0 | | | turbid | | | | | | | | | |
| 10 mM Citrate | 15 | 20 | white | 1 | 100 | dissolved | 0.5 | 200 | dissolved | 3.5 | 28.6 | dissolved |
| pH 6.0 | | | turbid | | | | | | | | | |
| 10 mM | 15 | 20 | white | 1 | 100 | dissolved | 0.5 | 200 | dissolved | 4 | 25 | dissolved |
| Phosphate | | | turbid | | | | | | | | | |
| pH 7.0 | | | | | | | | | | | | |
| 10 mM | 15 | 20 | white | 1 | 100 | dissolved | 0.5 | 200 | dissolved | 4 | 25 | dissolved |
| Phosphate | | | turbid | | | | | | | | | |
| pH 7.5 | | | | | | | | | | | | |
| 10 mM | 15 | 20 | white | 1 | 100 | dissolved | 0.5 | 200 | dissolved | 4 | 25 | dissolved |
| Phosphate | | | turbid | | | | | | | | | |
| pH 8.0 | | | | | | | | | | | | |

From the results shown in Table 1, it was found that the peptides of SEQ ID NOs: 2 to 4 could be dissolved at least in the range of 20 mg/mL to 200 mg/mL. The peptide of SEQ ID NO: 1, however, could not be dissolved under any of the conditions at the final concentration of 20 mg/mL.

### (Test Example 2) Examination of Solubility of SEQ ID NO: 1

Since the peptide of SEQ ID NO: 1 could not be dissolved in Test Example 1, additional conditions were examined for the peptide of SEQ ID NO: 1.

The solubility of the peptide drug substance of SEQ ID NO: 1 (purity 95 w/w % or more, PPL) was examined as described below. First, 10 to 500 mM glycine-HCl buffers containing 270 mM trehalose from pH 2.5 to 3.5 were prepared, and the peptide drug substance was added to 2 mL of each buffer so that the peptide concentration was 10 to 25 mg/mL. The mixture was stirred and allowed to stand at room temperature for 24 hours, and the solubility of the peptide and the pH of the peptide solution after dissolution were evaluated at room temperature (25° C.). The results are shown in Table 2.

TABLE 2

| | | | | final | | mass ratio |
|---|---|---|---|---|---|---|
| | | | | conc. of | | peptide |
| | glycine | | pH after | peptide | | (mg)/ |
| pH | mM | mg/mL | dissolution | (mg/mL) | result | glycine (mg) |
| 2.5 | 10 | 0.75 | 3.86 | 20 | dissolved | 26.67 |
| 3 | 10 | 0.75 | 4.10 | 20 | dissolved | 26.67 |
| 3.5 | 10 | 0.75 | 4.22 | 20 | dissolved | 26.67 |
| 3.5 | 50 | 3.75 | 3.8 | 10 | dissolved | 2.67 |
| 3.5 | 50 | 3.75 | 4.08 | 25 | white turbid | 6.67 |
| 3.5 | 100 | 7.5 | 3.7 | 10 | dissolved | 1.33 |
| 3.5 | 100 | 7.5 | 3.92 | 25 | white turbid | 3.33 |
| 3.5 | 200 | 15 | 3.65 | 10 | dissolved | 0.67 |
| 3.5 | 300 | 22.5 | 3.57 | 10 | dissolved | 0.44 |
| 3.5 | 500 | 37.5 | 3.61 | 25 | white turbid | 0.67 |

From the results shown in Table 2, it was found that the peptide of SEQ ID NO: 1 could be dissolved by using a glycine-HCl buffer, but the peptide could not be dissolved at a peptide concentration of 25 mg/mL even when the glycine concentration was 500 mM, which was the highest concentration of glycine, and thus the preferable peptide concentration was 20 mg/mL or less. It was also found that the peptide could be dissolved if the mass ratio of the peptide to glycine (peptide/glycine) was in the range of 0.44 to 26.67. In addition, it was found that the peptide could be stably dissolved if the pH after dissolution was in the range of 3.57 to 4.22.

### (Test Example 3) Evaluation of Peptide Stability

Based on the results of Test Example 1 and Test Example 2, the stability of the peptides was examined. First, peptide drug substances (purity 95 w/w % or more, PPL) were dissolved in the buffers shown in Table 3, respectively, to prepare peptide solutions of the peptides of SEQ ID NOs: 1 to 4 at the final concentration of 10 mg/mL. The peptide solutions were stored at 25° C./60% humidity or 40° C./75% humidity for 24 hours or 48 hours to evaluate the stability of the peptides. In the evaluation, samples were collected at the time of preparation of the peptide solution, after 24 hours, and after 48 hours, and stored at −20° C. The peptide concentration was measured by HPLC after all the samples were collected, and compared with the peptide concentration at the time of preparation (T=0). The results are shown in Table 3.

TABLE 3

| | | | T = 24 h | | T = 24 h | | T = 48 h | | T = 48 h | |
|---|---|---|---|---|---|---|---|---|---|---|
| | | | 25° C./60% RH | | 40° C./75% RH | | 25° C./60% RH | | 40° C./75% RH | |
| | | T0 | | % | | % | | % | | % |
| | | Conc. | Conc. | difference | Conc. | difference | Conc. | difference | Conc. | difference |
| peptide | buffer | mg/mL | mg/mL | vs T = 0 | mg/mL | vs T = 0 | mg/mL | vs T = 0 | mg/mL | vs T = 0 |
| SEQ ID | pH of 3.5 in 50 mM | 11.26 | 11.44 | 2 | 11.04 | −2 | 11.18 | −1 | 11.23 | 0 |
| NO: 1 | Gly-HCl buffer with | | | | | | | | | |
| | 200 mM Trehalose | | | | | | | | | |
| SEQ ID | pH of 4.0 in 10 mM | 10.89 | 10.85 | 0 | 10.24 | −6 | 10.37 | −5 | 8.76 | −20 |
| NO: 2 | Citrate buffer with | | | | | | | | | |
| | 270 mM Trehalose | | | | | | | | | |
| SEQ ID | pH of 7.0 in 10 mM | 11.23 | 11.42 | 2 | 11.70 | 4 | 11.63 | 4 | 11.74 | 4 |
| NO: 3 | Citrate buffer with | | | | | | | | | |
| | 270 mM Trehalose | | | | | | | | | |
| SEQ ID | pH of 6.0 in 10 mM | 10.61 | 10.47 | −1 | 10.68 | 1 | 10.16 | −4 | 10.47 | −1 |
| NO: 4 | Citrate buffer with | | | | | | | | | |
| | 270 mM Trehalose | | | | | | | | | |

From the results shown in Table 3, these conditions were found to be appropriate since the degradation of the peptides was suppressed to within 6% except when the peptide of SEQ ID NO: 2 was stored at 40° C./75% humidity for 48 hours. When the peptide of SEQ ID NO: 2 was stored at 4° C./75% humidity for 48 hours, 20% was degraded. Therefore, it was found that when preparing a cocktail formulation of the peptides of SEQ ID NOs: 1 to 4, it was better to prepare at 25° C./60% humidity or less within 48 hours or at 40° C./75% humidity within 24 hours.

### (Preparation Example 1) Production of Lyophilized Preparations for Preparing Peptide Cocktail Preparation

The peptide drug substances of SEQ ID NOs: 1 to 4 were added to 50 mM glycine-HCl buffer (pH 3.5) containing 200 mM trehalose, 50 mM citrate buffer (pH 4) containing 270 mM trehalose, 10 mM citrate buffer (pH 7) containing 270 mM trehalose, and 10 mM citrate buffer (pH 6) containing 270 mM trehalose, respectively, and pH of the solutions was adjusted with HCl and NaOH to provide peptide solutions having the formulations shown in Table 4 below. After filled into a vial at 10 mg per vial, each peptide solution was lyophilized to provide a lyophilized peptide preparation.

TABLE 4

| vial | 1 | 2 | 3 | 4 |
|---|---|---|---|---|
| peptide | SEQ ID NO: 1 | SEQ ID NO: 2 | SEQ ID NO: 3 | SEQ ID NO: 4 |
| | 10.00 mg/mL | 10.00 mg/mL | 10.00 mg/mL | 10.00 mg/mL |
| citric acid | — | 50 mM | 10 mM | 10 mM |
| | | (9.61 mg/mL) | (1.92 mg/mL) | (1.92 mg/mL) |
| glycine | 50 mM* | | | |
| | (3.75 mg/mL) | | | |
| trehalose | 200 mM | 270 mM | 270 mM | 270 mM |
| | (68.46 mg/mL) | (92.4 mg/mL) | (92.4 mg/mL) | (92.4 mg/mL) |
| pH | 3.5 | 4 | 7 | 6 |

*Mass ratio of the peptide of SEQ ID NO: 1 to grycine = 2.67

### Example 1

Using the lyophilized peptide preparations produced in Preparation Example 1, a peptide cocktail preparation to be administered was prepared as follows. First, 1 mL of purified water was added to each lyophilized peptide preparation, and the lyophilized product in the vial was dissolved to prepare a peptide solution. All the peptide solutions were collected from the respective vials and transferred to a different vial to provide a peptide cocktail preparation. Also, a GM-CSF lyophilized preparation (product name: Lukine (250 mg/vial), Sanofi) was dissolved in 1 mL of purified water and 0.6 mL of the solution thus obtained was added as an adjuvant to 1.4 mL of the peptide cocktail preparation to provide a peptide cocktail preparation to be administered. Then, the peptide cocktail preparation thus obtained was allowed to stand at room temperature for 6 hours, and evaluated for changes in peptide concentration, pH (25° C.), and osmotic pressure compared with those before standing. The results are shown in Table 5.

TABLE 5

| analysis item | T = 0 hour | T = 6 hours |
|---|---|---|
| adjustment time | 25 minutes | |
| appearance | clear aqueous solution | clear aqueous solution |
| (pooled sample) | | |
| pH | 4.9 | 4.9 |
| osmotic pressure | 503 mOsm/Kg | 495 mOsm/Kg |
| HPLC analysis | SEQ ID NO: 1: 2.6 mg/mL | SEQ ID NO: 1: 3.0 mg/mL (113.1% vs T0) |
| | SEQ ID NO: 2: 2.7 mg/mL | SEQ ID NO: 2: 2.8 mg/mL (107.0% vs T0) |
| | SEQ ID NO: 3: 2.5 mg/mL | SEQ ID NO: 3: 2.7 mg/mL (107.7% vs T0) |
| | SEQ ID NO: 4: 2.8 mg/mL | SEQ ID NO: 4: 3.0 mg/mL (106.4% vs T0) |

From the results shown in Table 5, it was found that the peptide of SEQ ID NO: 1 was stable without precipitation even at pH 4.9. From these results as well as the results of Test Example 2, it was found that the peptide of SEQ ID NO: 1 could be stably dissolved at pH 3.5 to 5.0.

### Example 2

To study the formulation of a peptide cocktail preparation, the peptide drug substances of SEQ ID NOs: 1 to 4 (purity 95 w/w % or more, PPL) were added to 250 ml of 50 mM (3.75 mg/mL) glycine-HCl buffer (pH 3.5) containing 100 mg/mL trehalose so that the concentration of each peptide was 3 mg/mL (mass ratio of the peptide of SEQ ID NO: 1 to glycine was 0.80), and the pH of the solution was adjusted to 3.5 with HCl and NaOH at room temperature (25° C.) and stirred at room temperature for 24 hours to provide a peptide cocktail preparation. After the stirring, appearance of the solution was observed to evaluate the stability after dissolution of the peptides. The result is shown in Table 6.

### Comparative Example 1

A peptide cocktail preparation was prepared in the same manner as in Example 2 except that 25 mM citrate buffer was used instead of the 50 m M glycine-HCl buffer of Example 2, the final concentration of the peptide of SEQ ID NO: 1 was 4.5 mg/mL, and the pH of the solution after the peptides were dissolved was adjusted to 4.5. Appearance of the solution was observed to evaluate the solubility of the peptides. The result is shown in Table 6.

### Comparative Example 2

A peptide cocktail preparation was prepared in the same manner as in Example 2 except that 25 mM citrate buffer was used instead of the 50 mM glycine-HCl buffer of Example 2 and the pH of the solution after the peptides were dissolved was adjusted to 4.5. Appearance of the solution was observed to evaluate the solubility of the peptides. The result is shown in Table 6.

TABLE 6

| | Example 2 | Comparative example 1 | Comparative example 2 |
|---|---|---|---|
| peptide | SEQ ID NO: 1: 3 mg/mL | SEQ ID NO: 1: 4.5 mg/mL | SEQ ID NO: 1: 3 mg/mL |
| | SEQ ID NO: 2: 3 mg/mL | SEQ ID NO: 2: 3 mg/mL | SEQ ID NO: 2: 3 mg/mL |
| | SEQ ID NO: 3: 3 mg/mL | SEQ ID NO: 3: 3 mg/mL | SEQ ID NO: 3: 3 mg/mL |
| | SEQ ID NO: 4: 3 mg/mL | SEQ ID NO: 4: 3 mg/mL | SEQ ID NO: 4: 3 mg/mL |
| buffer | 100 mg/mL trehalose + | 100 mg/mL trehalose + | 100 mg/mL trehalose + |
| | 50 mM (3.75 mg/mL) glycine buffer | 25 mM(4.8 mg/mL) citrate buffer | 25 mM(4.8 mg/mL) citrate buffer |
| pH | 3.5 | 4.5 | 5.0 |
| appearance | clear aqueous solution | milky white aqueous solution | milky white aqueous solution |
| after 24 hours | | | |

As shown in Table 6, the peptides were stably dissolved in the preparation of Example 2, which used 50 mM glycine-HCl buffer, whereas precipitation was observed in the preparations of Comparative Examples 1 and 2. From these results and the results of Test Example 1, Test Example 2, and Example 1, it was found to be preferable that the peptide concentration of SEQ ID NO: 1 was 20 mg/mL or less, preferably 3 mg to 20 mg/mL, more preferably 3 mg to 10 mg, the mass ratio of the peptide to glycine was 0.3 to 30, preferably 0.6 to 3.0, and pH was 3.5 to 5.0.

### Preparation Example 2

To produce a lyophilized peptide cocktail preparation and confirm the stability of the preparation, 500 mL of a peptide cocktail preparation was prepared in the same manner as in Example 1, and then the preparation was aseptically filled to a glass vial at 1 mL per vial and lyophilized to provide a lyophilized peptide cocktail preparation. Then, immediately after the preparation (T=0) or after storage at room temperature (25° C., humidity 60%) for 1 month from the preparation (T=1), 1 mL of purified water was added to the lyophilized preparation to dissolve the peptides. Appearance, solubility, peptide stability, and pH were evaluated. The results are shown in Table 7.

TABLE 7

| time | T = 0 | T = 1 |
|---|---|---|
| appearance | white cake | white cake |
| appearance after dissolution | clear aqueous | clear aqueous |
| in 1 mL purified water | solution | solution |
| change in peptide amount | — | SEQ ID NO: 1: 99.5% |
| % vs T = 0 (100%) | | SEQ ID NO: 2: 100.4% |
| (HPLC analysis) | | SEQ ID NO: 3: 104.5% |
| | | SEQ ID NO: 4: 101.2% |
| pH | 3.5 | 3.5 |

It was found that the lyophilized preparation obtained by lyophilizing the peptide cocktail preparation of Example 1 did not show precipitation when dissolved with purified water and the peptides could be stably stored at room temperature.

### Example 3

A human clinical trial was carried out using the lyophilized peptide preparation produced in Preparation Example 1.

### Patients

The subjects who participated in this clinical trial were HLA-A2-positive patients with malignant melanoma.

### Drugs

The lyophilized preparations comprising the four peptides produced in Preparation Example 1 were used in this clinical study.

### Peptide Administration

A peptide cocktail preparation was prepared using the lyophilized preparations of the peptides of SEQ ID NOs: 1 to 4 in the same manner as in Example 1. The prepared peptide cocktail preparation was intradermally administered at a dose of 0.1 mg/peptide, 1.0 mg/peptide, or 3 mg/peptide per administration to 6 patients for each dose. The peptide administration was carried out weekly from the start of administration to the 4th administration and every 4 weeks after the 5th administration, and 8 times at a maximum.

### Safety Assessment

The safety was evaluated according to the criteria of Common Terminology Criteria for Adverse Events 4.0 (CTCAE 4.0) by observing adverse events that occurred after administration of the peptide cocktail preparation.

### Measurement of CTL Activity

Peripheral blood mononuclear cells (PBMCs) were isolated from peripheral blood collected after 4 or 8 times administration of the peptide cocktail preparation, and then cryopreserved. After the peptide administration was completed, all the collected PBMCs were examined for CTL activity by a method previously described (Hida N, Maeda Y, Katagiri K, Takasu H, Harada M, Itoh K. A simple culture protocol to detect peptide-specific cytotoxic T lymphocyte precursors in circulation. Cancer Immunol Immunotherapy 2002; 51:219-228.). First, PBMCs were cultured for 2 weeks in the presence of each administered peptide, and the PBMCs were co-cultured with HLA-A2-positive target cells presenting the administered peptide via the HLA molecule. Then, IFN-γ secreted when CTLs in the PBMCs reacted with the target cells was measured by ELISPOT method and the number of IFN-γ secreting cells was counted. The co-culture was carried out in three independent cultures for each peptide to obtain three values for each peptide from PBMCs obtained by each blood collection. The difference between the average value of these three cell numbers and that of three cell numbers obtained by co-culture with target cells not presenting the peptide (referred to as S), and the difference between the average value of these three cell numbers and that of three cell numbers obtained in the same way using a negative control peptide (HIV-derived peptide) instead of the administered peptide (referred to as N) were used, and the case where the S/N value was 2 or more and the S-N value was 10 or more was judged to be positive. The results are shown in Table 8.

TABLE 8

| | After 4 times | After 8 times |
|---|---|---|
| dose | administration | administration |
| 0.1 mg/peptide | 0 | 33% |
| | (0/3) | (1/3) |
| 1.0 mg/peptide | 33% | 50% |
| | (1/3) | (2/4) |
| 3.0 mg/peptide | 0 | 33% |
| | (0/4) | (1/3) |

Positive case(s)/examined case(s) is shown in parentheses.

From the results shown in Table 8, it was found that positive reaction to the peptides was obtained after 4 or 8 times administration at any dose. Also, the response tended to be better when 1.0 mg or more of the peptide was administered. Regarding safety, grade 3 facial edema, pruritus and urticaria were observed in one patient who received 0.1 mg of peptide, and grade 3 headache was observed in one patient who received 1.0 mg of peptide. However, other events were grade 2 or lower, and although injection site reactions were observed in all cases, adverse events observed were all within the acceptable range of safety.

### Example 4

A human clinical trial was carried out using the lyophilized peptide preparations produced in Preparation Example 1.

### Patients

The subjects who participated in this clinical trial were HLA-A2-positive lung cancer patients and they were non-small cell lung cancer patients who were determined to have metastases for the first time.

### Drugs

The lyophilized peptide preparations produced in Preparation Example 1 and pembrolizumab (Merck & Co., Inc.) were used in this clinical study.

### Administration of Peptides and Pembrolizumab

A peptide cocktail preparation was prepared using the lyophilized preparations of the peptides of SEQ ID NOs: 1 to 4 in the same manner as in Example 1. The prepared peptide cocktail preparation was administered intradermally to the patients at a dose of 3 mg/peptide (administration volume 1 mL). The peptide administration was carried out weekly from the start of administration to the 4th administration, and every 3 weeks after the 5th administration, and 16 times at a maximum. Administration of pembrolizumab was started on the same day as the administration of the peptide cocktail preparation, and the administration was carried out once every 3 weeks at a maximum of 35 cycles.

### Evaluation of Clinical Effects

The lesion to be subjected to image evaluation was set by CT or MRI examination and the tumor size of the evaluable lesion (Best Target Lesion Response) was evaluated to determine PR (partial response), SD (stable disease), or PD (progressive disease). Evaluation was carried out every 9 to 12 weeks from the start of administration. In this study, the change ratio in tumor size was evaluated on a pre-administration basis in 7 patients whose clinical efficacy could be evaluated.

### Measurement of CTL Activity

Of the 7 patients whose clinical efficacy was evaluated (as of the end of May 2020), CTL activity was measured and evaluated in 3 patients who completed blood sampling at the start of administration, after 8 times administration, and after 16 times administration. Peripheral blood mononuclear cells (PBMCs) were isolated from collected peripheral blood and cryopreserved, and after the administration was completed, all the PBMCs were examined for CTL activity. The measurement was carried out by a known ELISPOT method. Specifically, after the cryopreserved PBMCs were thawed, the cells were seeded on an ELISPOT plate at 4×10⁵ cells/well, and serum-free medium (CTL serum free test medium, 1% L-glutamine, CTL) containing the peptides at the final concentration of each peptide of 100 μg/mL was added to the PBMCs for stimulation overnight. After the stimulation, the cell supernatant and cells were removed, and IFN-γ secreted from the cells and trapped on the plate was stained with an HRP-labeled anti-IFN-γ antibody and analyzed with CTL ImmunoSpot® Analyzer (CTL) to measure the number of IFN-γ secreting cells.

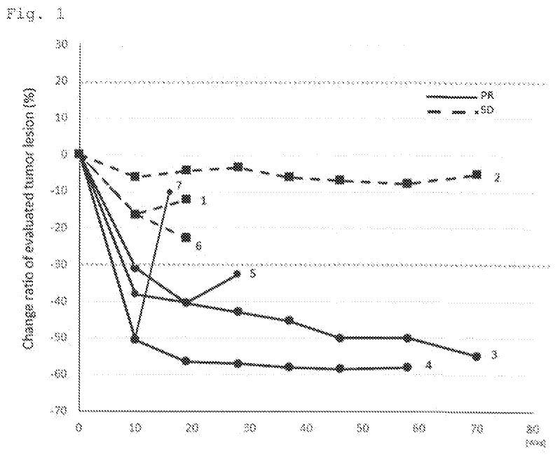

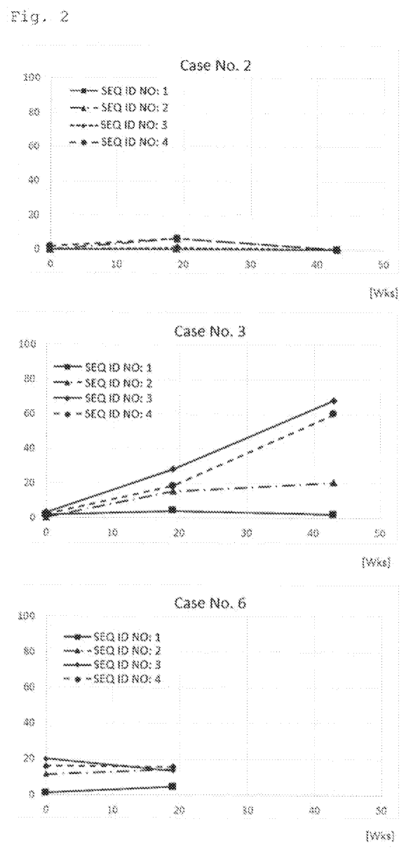

The clinical effects are shown in FIG. 1 and the CTL activity is shown in FIG. 2. From FIG. 1, it was found that as a result of the combined administration of the peptide cocktail preparation and pembrolizumab, 4 cases had PR and 3 cases had SD, and the response rate was 57% (4/7 cases). The response rate of pembrolizumab alone was 45% in similar subjects (Reck, M., et al., Pembrolizumab versus Chemotherapy for PD-L1-Positive Non-Small-Cell Lung Cancer. N Engl J Med, 2016, 375(19): p. 1823-1833), suggesting that the combination use of the peptide cocktail preparation and pembrolizumab could provide an excellent clinical effect. Also, in the PR case among the 3 cases for which CTLs could be measured, the number of IFN-γ-producing cells that responded to the peptide increased as the number of peptide administrations increased (FIG. 2, middle), indicating that the immune response was enhanced by administration of the peptide cocktail preparation. These results suggests that the clinical effects are related to the immune response to the peptide cocktail preparation.

### Example 5

The peptide drug substances of SEQ ID NOs: 1 to 4 (purity 95 w/w % or more, PPL) were dissolved with 50 mM (3.75 mg/mL) glycine-HCl buffer (pH 3) so that each peptide concentration was 3 mg/mL (mass ratio of the peptide of SEQ ID NO: 1 to glycine was 0.80) to prepare a peptide cocktail. To the peptide cocktail, 7% IV Meylon (7% sodium hydrogen carbonate, Otsuka Pharmaceutical Factory), 1N sodium hydroxide, 200 mM phosphate buffer (pH 8.0) or 200 mM histidine buffer (pH 6.5) was added to raise and adjust pH to a near-neutral pH. The solubility was evaluated by observing appearance of the solution immediately after the pH adjustment and after 24 hours. The results are shown in Table 9.

TABLE 9

| | pH adjuster | | | |
|---|---|---|---|---|
| | | | 200 mM | 200 mM |
| | | 1N sodium | phosphate buffer | histidine buffer |
| | 7% IV Meylon | hydroxide | (pH 8.0) | (pH 6.5) |
| pH after adjustment | 7.37 | 8.068 | 6.927 | 6.771 |
| appearance | clear | clear | clear | clear |
| (immediately after pH | | | | |
| adjustment) | | | | |
| appearance | clear | clear | clear | clear |
| (after 24 hours) | | | | |

From the results shown in Table 9, it was found that the peptide cocktail comprising the peptides of SEQ ID NOs: 1 to 4 dissolved with 50 mM glycine-HCl buffer (pH 3.5) stably maintained the dissolved state even at a near-neutral pH.

1. A cancer peptide vaccine comprising a peptide of Asn-Val-Leu-His-Phe-Phe-Asn-Ala-Pro-Leu (SEQ ID NO: 1), a peptide of Ala-Ser-Leu-Asp-Ser-Asp-Pro-Trp-Val (SEQ ID NO: 2), a peptide of Lys-Leu-Lys-His-Tyr-Gly-Pro-Gly-Trp-Val (SEQ ID NO: 3), and a peptide of Leu-Leu-Gln-Ala-Glu-Ala-Pro-Arg-Leu (SEQ ID NO: 4).

2. The cancer peptide vaccine according to claim 1, further comprising glycine.

3. The cancer peptide vaccine according to claim 1, wherein the mass ratio of the peptide of SEQ ID NO: 1 to glycine is 0.3 to 30.

4. The cancer peptide vaccine according to claim 1, which is a lyophilized preparation.

5. The cancer peptide vaccine according to claim 1, which is used in combination with an immune checkpoint inhibitor.

6. The cancer peptide vaccine according to claim 5, wherein the immune checkpoint inhibitor is an anti-PD-1 antibody.

7. The cancer peptide vaccine according to claim 6, wherein the anti-PD-1 antibody is pembrolizumab.

8. A method of preparing a cancer peptide vaccine, comprising adding a peptide of Asn-Val-Leu-His-Phe-Phe-Asn-Ala-Pro-Leu (SEQ ID NO: 1) to an aqueous solution comprising glycine, wherein the cancer peptide vaccine comprises the peptide of Asn-Val-Leu-His-Phe-Phe-Asn-Ala-Pro-Leu (SEQ ID NO: 1), a peptide of Ala-Ser-Leu-Asp-Ser-Asp-Pro-Trp-Val (SEQ ID NO: 2), a peptide of Lys-Leu-Lys-His-Tyr-Gly-Pro-Gly-Trp-Val (SEQ ID NO: 3), and a peptide of Leu-Leu-Gln-Ala-Glu-Ala-Pro-Arg-Leu (SEQ ID NO: 4).

9. The method according to claim 8, wherein the peptide of SEQ ID NO: 1 is added to the aqueous solution comprising glycine so as to provide an aqueous solution having the mass ratio of the peptide of SEQ ID NO: 1 to glycine of 0.3 to 30.

10. The method according to claim 8, wherein the aqueous solution comprising glycine has a pH of 3.5 to 5.0.

11. The method according to claim 8, wherein the peptide of SEQ ID NO: 1 is added to an aqueous solution comprising glycine so as to provide an aqueous solution having a peptide concentration of 20 mg/mL or less.

12. The method according to claim 8, which comprises providing an aqueous solution comprising the peptide of SEQ ID NO: 1, an aqueous solution comprising the peptide of SEQ ID NO: 2, an aqueous solution comprising the peptide of SEQ ID NO: 3, and an aqueous solution comprising the peptide of SEQ ID NO: 4, separately.

13. The method according to claim 12, further comprising lyophilizing each of the aqueous solutions.

14. The method according to claim 8, which comprises providing an aqueous solution comprising the peptide of SEQ ID NO: 1, the peptide of SEQ ID NO: 2, the peptide of SEQ ID NO: 3, and the peptide of SEQ ID NO: 4.

15. The method according to claim 14, comprising adjusting the pH of the aqueous solution comprising the peptides to a pH of 3.5 to 8.2.

16. The method according to claim 14, further comprising lyophilizing the aqueous solution.
